(19) United States
(12) Patent Application Publication
(10) Pub. No.: US 2006/0105335 A1
(43) Pub. Date: May 18, 2006

(54) Color coated layer-by-layer microcapsules serving as combinatory analysis libraries and as specific optical sensors

(76) Inventors: Lars Daehne, Berlin (DE); Barbara Baude, Caputh (DE); Andreas Voigt, Lindenst (DE)

Correspondence Address: Moser Patterson & Sheridan;Zimmerman & Partner, Patent Counsel, Suite 1500 3040 Post Oak Boulevard, Houston, TX, 77056-6582, US

(21) Appl. No.: 10/522,998

(22) PCT Filed: Jul. 29, 2003
(86) PCT No.: PCT/EP03/08376

§ 371 Date: Aug. 11, 2005

(30) Foreign Application Priority Data
Aug. 2, 2002 (DE) 10236409.5
Apr. 2, 2003 (DE) 10315846.4

(51) Int. Cl. C12Q 1/68; G01N 33/53; C12M 1/34; C40B 40/10

(52) U.S. Cl. 435/6; 435/7.1; 435/287.2; 977/924

(57) ABSTRACT

Monodisperse colloids were coated with polyelectrolytes using the layer-by-layer method. The template cores can remain in the interior or be dissolved away. Various fluorescent dyes are covalently bonded, in defined quantity, to the polyelectrolytes. The quantity of dye is controlled by varying the label content or by coprecipitating unlabeled polymers. Different. dye layers are separated from each other by intermediate layers, resulting in unwanted interactions being suppressed. Conversely, a FRET signal can be generated between suitable dye pairs at short distances (0-6 nm), with it being possible to control this signal independently of the dye concentration by means of the number of intermediate layers. The capsule coding is read out by varying the excitation and emission wavelengths. Macromolecules which fish out complementary substances from solutions can be immobilized in the capsules. Particles which are coated in this way, or hollow capsules, can be used as sensors after a sensitive intermediate layer has been introduced. Changes in the size/structure of the intermediate layer can be detected either by FRET occurring between adjacent, labeled polyelectrolyte layers or by self-quenching/aggregate fluorescence of dyes in the sensitive layer.

The present invention relates to combinatorial libraries which are based on hollow or filled polyelectrolyte capsules which are prepared by the layer-by-layer method. The LbL method makes it possible to control the number and the concentration, and the distance between the dye molecules on the nanometer scale, resulting in a higher quantity of coded information in the wall (envelope) than is known to be possessed by particles (beads, solid microparticles) which are color-coded in their volume or at their surface. Furthermore, the fluorescent dye is entirely concentrated at the surface, something which is advantageous for FRET-based detection in homogeneous particle assays since the high background fluorescence of the dyes which are located in the interior of the particle, and which do not, therefore, participate in the FRET, is entirely absent.¹³ The second part of the invention deals with the possibility of filling capsules with different macromolecules while still keeping the capsules permeable to small molecules. Color-coded capsules of this nature can be used as combinatorial capturing receptacles which are able to take up a substantial quantity of specific substances from a reaction mixture. Subsequently, the different capsules, containing different substances in their interior, can be sorted on the basis of their specific fluorescence signals. These combinatorial libraries can be used in many fields in medicine, biology and chemistry.

There is a limit to the extent to which assays and microtiter plates can be miniaturized with a view to increasing assay capacity still further. The libraries which are based on beads open up the possibility of an alternative method. New developments in flow cytometry (e.g. COPAS™ bead flow sorting) make it possible to achieve a throughput of up to 100 000 particles per second. For this reason, the libraries which are based on beads could become the leading technology in screening or collecting operations.^{1-5,7}

We have prepared hollow capsules from poly-electrolytes,⁶ with the capsules containing different color combinations in their walls. While the color-coded capsules can be sorted like beads, they are hollow and can possess many binding sites both on the wall surface and in their interior.

These capsules possess a variety of advantages as compared with the beads technology:
1. Their mass is very small. They therefore sediment out of solutions of differing density substantially more slowly than do beads.
2. As a consequence of their thin wall, and the same or similar material being present in the interior as in the exterior, light scattering is very low. In the case of beads, differences in the refractive index between the bead and the solvent (usually water) lead to a high degree of light scattering, with this impairing the sorting process in the flow cytometer.
3. Reactions can only take place at the surface of the beads. The number of binding sites possessed by the beads is therefore very limited. In the case of our capsules, the external wall surface, the internal wall surface, and the entire volume, of the capsules can be used for reactions. A capsule (or a bead) having a diameter of 5 μm has an outer surface of 78 μm² and a volume of 65 μm³. Assuming a binding-site concentration of 0.1 M, a bead only has about 9×10⁴ binding sites whereas a capsule possesses about 5000 times more binding sites, namely 4×10⁸ binding sites.
4. The dye labels can be applied at a distance from each other which is adequate for avoiding interactions such as the formation of H aggregates or J aggregates, self-quenching or Förster resonance energy transfer, all of which interfere with the fluorescence signals when the solid body phase is labeled with different dyes. This allows more combinatorial possibilities.
5. Förster resonance energy transfer signals can be set in a controlled manner for the purpose of ensuring more forgery-proof coding of trademarks, i.e. for labeling the product which is provided with the trademark.
6. The internal space of the capsules can be filled with highly active bioactive compounds such as enzymes, DNA or the like, or with specifically functionalized polyelectrolytes, which enable coreactants to be selectively captured from solution by means of bioreactions, physisorption or chemisorption. The coded capsules can then subsequently be sorted.
7. The coded information can be set by the number of the dyes and their relationship to each other, and by distance-dependent interactions between the dyes, as, for example, the Förster resonance energy transfer. In the case of the known fluorescent beads,⁴ such interactions are undesirable since it is not possible to control the distances between the dye molecules.
8. Hollow coded capsules can be prepared and their internal space can be used for immobilizing macromolecules (polyelectrolytes, proteins and enzymes). The functionalized macromolecules can fish out complementary compounds from reaction solutions by means of physisorption, chemisorption or biological bonding.

The present invention relates to sensors which are constructed, by means of the layer-by-layer (LbL) method, on colloids having diameters of less than 100 μm and which react to chemical substances or physical parameters. Where appropriate, the colloidal template can be leached out in a following step, such that hollow capsules are formed.

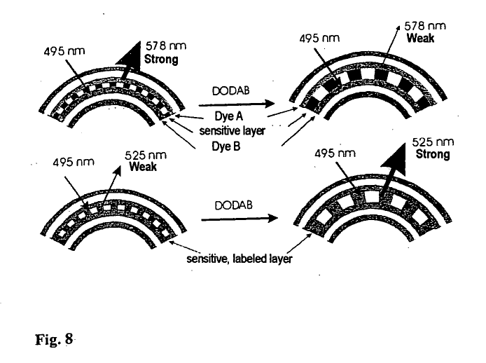

The sensor effect is achieved by means of a layer of defined thickness composed of a special material which either swells or shrinks when the concentration of a substance in the surrounding solution is altered or when physical parameters are changed. The emission of fluorescent dyes is used for detecting this process. Two variants of the mode of action are possible (FIG. 8):

1. The sensitive layer, having a thickness of between 0.1 nm and 10 nm, is located between two layers composed of polyelectrolytes. The polyelectrolyte layer on one side of the sensitive layer contains a firmly integrated fluorescent dye of higher absorption energy (donor) while the polyelectrolyte layer on the other side contains a fluorescent dye of lower absorption energy (acceptor). Emitting nanoparticles can also be used instead of fluorescent dyes. The dye pair is coordinated such that a Förster (fluorescence) resonance energy transfer (FRET) takes place. The efficiency of the FRET depends sensitively on the distance of the dye molecules from each other. The FRET signal can be detected spectrometrically in a static manner using either the donor fluorescence or the acceptor fluorescence or in a time-dependent manner using the donor fluorescence.

2. The sensitive material is linked covalently, at comparatively high concentration, to a fluorescent dye (mass of material:mass of dye <500:1). The dye is distinguished by the fact that it readily forms dimers/aggregates with itself. If the labeled material is introduced into a capsule wall as at least one homogeneous layer having a thickness of from 1 nm to 1 μm, a self-quenching process in connection with the formation of dimers or H aggregates leads to the fluorescence of the dye monomers being quenched whereas a new emission band at lower energy arises when J aggregates or excimers are formed. When the layer in the capsule wall swells/shrinks, the signal can be detected by way of the intensity or lifetime of the monomer fluorescence or by way of the ratio of monomer fluorescence to the fluorescence of the J aggregate or excimers.

In general, the capsules according to the invention, which preferably have a diameter of less than 100 μm, possess an envelope which is composed of at least three polyelectrolyte layers, with one of the three polyelectrolyte layers being labeled with at least one dye. This dye, which can be a fluorescent dye or emitting (fluorescent) nanoparticles (particles having a size of preferably less than 1 nm), serves, for example, for identifying the capsules. In this case, the capsules are used for labeling or coding industrial products, particles, cells, tissues, organs or organisms of biological origin such that the provenance of the latter can be established and identified on the basis of the fluorescence of the dye. On the other hand, the capsules can also be used as sensors which react measurably to altered environmental conditions by altering the fluorescence of the dye. Finally, the capsules can also be used as “capturing receptacles” in order to remove substances from solutions and/or identify them. Capsules which are labeled with different dyes and which in each case react specifically with a different substance, for example by means of specific binding sites, are suitable for use as a library of reporter particles for identifying substances and/or labeling processes. It lies within the scope of the invention to combine these applications with each other.

Within the scope of the invention, “polyelectrolytes” are understood as being, in particular, water-soluble molecules or aggregates which carry at least 2 charges, preferably even at least three charges. Substantially more charges are even present in the case of many polyelectrolytes. Within the scope of the invention, the polyelectrolytes include, in particular, organic polyelectrolytes, nanoparticles, polyampholytes and compounds and complexes which are composed of organic polyelectrolytes and low molecular weight substances, e.g. surfactants.

The polyelectrolyte layers are, in particular, layers which essentially have the thickness of about one monolayer of the corresponding polyelectrolyte. Such polyelectrolyte layers can, for example, be applied using layer-by-layer methods. In these methods, polyelectrolytes of alternating polarity are applied, with polyelectrolytes accumulating on existing polyelectrolyte layers until the charges on the already existing layer are saturated.

Multilayer polyelectrolyte capsules, which can also consist of different polyelectrolyte layers, can be prepared, for example, in accordance with the layer-by-layer method which is described in DE 198 12 083 A1, DE 199 07 552 A1, EP 98 113 181, WO/47252 and U.S. Pat. No. 6,479,146, the entire disclosure content of which is hereby incorporated by reference.

Insofar as the capsules are used as sensors, two of the three envelope layers can, for example, in each case be labeled with a different dye. The third polyelectrolyte layer, which is not labeled with fluorescent dyes, then lies between the two labeled polyelectrolyte layers. As a result, the latter two layers are at a certain distance from each other, which distance corresponds approximately to the thickness, for example from 0.1 nm to 10 nm, of the unlabeled central third layer. In this connection, the thickness of the polyelectrolyte layer depends, inter alia, on the polyelectrolyte which is used. The dyes which are used are selected such that they exhibit different emission and absorption bands, with the emission band of one of the dyes at least partially overlapping the absorption band of the other dye. As a result, radiationless transfers, i.e. a FRET, can take place between the dyes. By this means, the dye possessing the higher absorption energy (acceptor) can pass on its excitation to the other dye (dye possessing lower absorption energy; donor) without the acceptor dye being observed to fluoresce. The radiationless transfer consequently leads to excitation of the donor dye, whose fluorescence can be measured. If the acceptor dye absorbs in the blue and fluoresces in the green, for example, the donor dye should then absorb in the green and, for example, emit in the red. An excitation with blue light then leads, in connection with a radiationless transfer between the dyes, to an observed fluorescence in the red instead of in the green. The efficiency of the radiationless transfer between the dye molecules depends heavily on the distance between the molecules, with this distance being determined by the thickness of the unlabeled third polyelectrolyte layer. If this thickness changes, for example as a reaction to altered environmental conditions, the strength of the coupling between the dye molecules then changes. It is therefore also possible to refer to the layer as being sensitive (sensory intermediate layer). If the distance between the dye molecules is small, a transfer which is virtually radiationless then takes place, i.e. only slight fluorescence of the acceptor dye, but relatively high fluorescence of the donor dye, can be detected. When the distance is increased, the fluorescence of the acceptor dye increases while that of the donor dye decreases. These changes can be measured and serve as a measure of the change in the layer thickness. The environmental conditions whose change leads to a change in the thickness of the unlabeled layer can be the pH, the salt concentration, the temperature, adsorbed components, enzymes, the concentration of a substance, physical parameters, components which affect the solvent or which react with the sensitive layer, and also miscible solvent constituents. Organic polyelectrolytes in particular react sensitively to altered environmental conditions. For example, a change in the temperature leads to a change in the ability of the organic polyelectrolytes to take up water and consequently to a change in the thickness of the layer. An example in this regard is PAH.

In addition to the unlabeled polyelectrolyte layer, further polyelectrolyte layers can be arranged between the dye-labeled polyelectrolyte layers, or else the unlabeled polyelectrolyte layer can itself consist of several polyelectrolyte layers.

However, sensory capsules can also only be labeled with one dye. In this case, the dye is bound, at high concentration, to sensitive material within a polyelectrolyte layer, with the sensitive material being able to react to the altered environmental conditions by an increase or decrease in volume. The high concentration of the dye leads to self-quenching, for example as the result of dimer formation, or to the generation of new emission bands when excimers are formed. In this case, too, these processes depend greatly on the distance between the dye molecules, such that a change in the thickness of the layer also leads to a change in the distance between the dye molecules.

When the capsules are used as “capturing receptacles”, they possess specific binding sites for the molecules which are to be captured. The binding sites can be located in the interior of the capsules or on their envelopes. Capsules possessing different binding sites can be labeled with different dyes such that it is then possible to subsequently sort the capsules on the basis of the fluorescence. In this way, it is possible to selectively isolate substances, e.g. proteins, from solutions.

### DESCRIPTION OF THE EXPERIMENTS

Labeling Polyelectrolytes with Dyes:

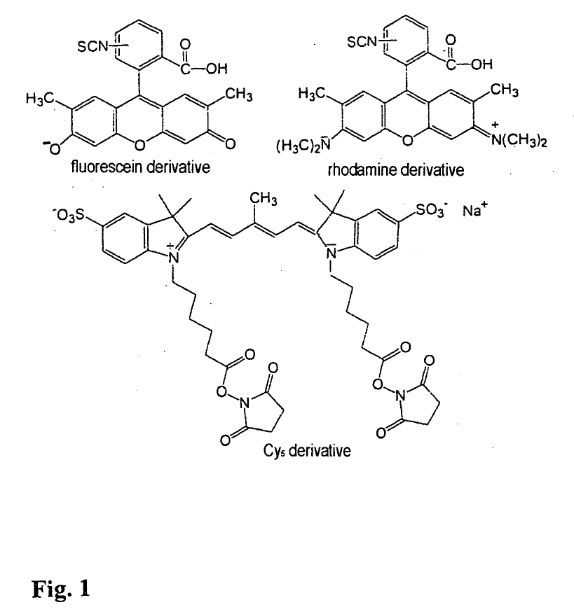

PAH was labeled with the dye derivatives fluorescein isothiocyante and tetramethylrhodamine isothiocyanate and a derivative of CY5. The formulae are depicted in FIG. 1. The labeling reactions were carried out in accordance with the general approach when labeling proteins. Instead of a hydrogen carbonate buffer, NaOH was used for activating approx. 30% of the PAH groups. The reaction mixture was dialyzed against water. After HCl had been added to the solution of labeled PAH in order to adjust the pH to 4-5, the solution was lyophilized. The labeled content was determined by means of UV/Vis spectroscopy and was 53:1 in the case of PAH-Fl, 580:1 in the case of PAH-Rho and 500:1 in the case of PAH-Cy5 (ratio of the PAH units:number of labeled molecules). The yield of label was approx. 80% in the case of fluorescein, 20% in the case of rhodamine and 40% in the case of Cy5. Each PAH was only labeled with one dye since simultaneously labeling a PAH chain has the potential disadvantage of giving rise to self-quenching or Förster resonance energy transfer.

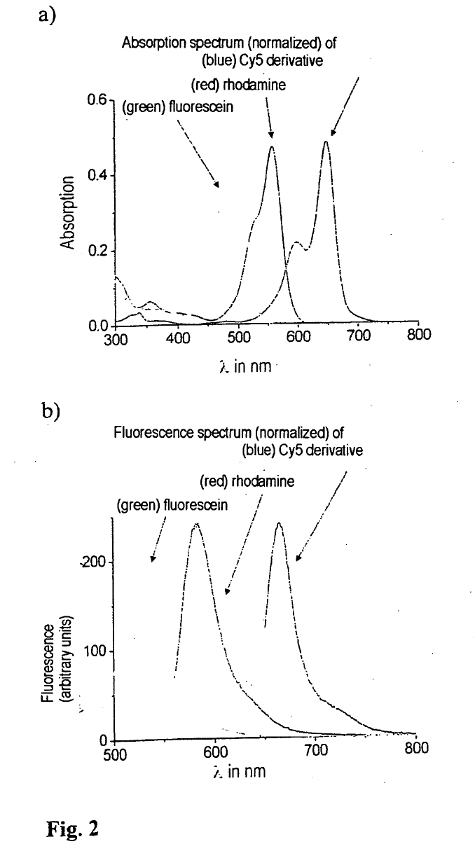

The absorption and fluorescence spectra of the dyes are shown in FIGS. 2a and b. The absorption maxima of the three labeled PAH polymers were determined as being 495, 557 and 648 nm. The fluorescence maxima were 520, 582 and 665 nm, with the absorption wavelength being used for the excitation.

Preparing the Capsules

Silica templates of 3 μm in size were coated with 10 alternating layers of poly(allylamine hydrochloride) (PAH, MW 60 000 g/mol) and poly(styrene sulfonate) (PSS, MW 70 000 g/mol).⁹ In order to obtain distinguishable walls, differently labeled PAH polymers were used for the coating. Only one layer of the given PAH was used for coloring the capsules. Only in the case of Cy5 were 2 layers used for the labeling; this was because of the lower fluorescence quantum yield and the low dye content. An attempt was made to maintain a certain distance between the different dye layers in order to avoid Förster resonance energy transfer. The following capsules were prepared:

TABLE 1
Dye-coded capsules containing different types of PAH-dye layers

| Layer/ | | | | | | | | |
|---|---|---|---|---|---|---|---|---|
| capsule | 1. | 2. | 3. | 4. | 5. | 6. | 7. | 8. |

| | | | | | | | | |
|---|---|---|---|---|---|---|---|---|
| 1. PAH | — | — | — | | — | — | — | — |
| 2. PSS | — | — | — | — | — | — | — | — |
| 3. PAH | — | — | — | Cy5 | Cy5 | Cy5 | Cy5 | — |
| 4. PSS | — | — | — | — | — | — | — | — |
| 5. PAH | Rho | Rho | Fluo | Cy5 | Cy5 | Cy5 | Cy5 | — |
| 6. PSS | — | — | — | — | — | — | — | — |
| 7. PAH | — | — | — | — | Fluo | Rho | Fluo | — |
| 8. PSS | — | — | — | — | — | — | — | — |
| 9. PAH | — | Fluo | — | — | — | — | Rho | — |
| 10. PSS | — | — | — | — | — | — | — | — |

Hollow capsules were obtained by leaching out the silica template with hydrofluoric acid and washing with water.

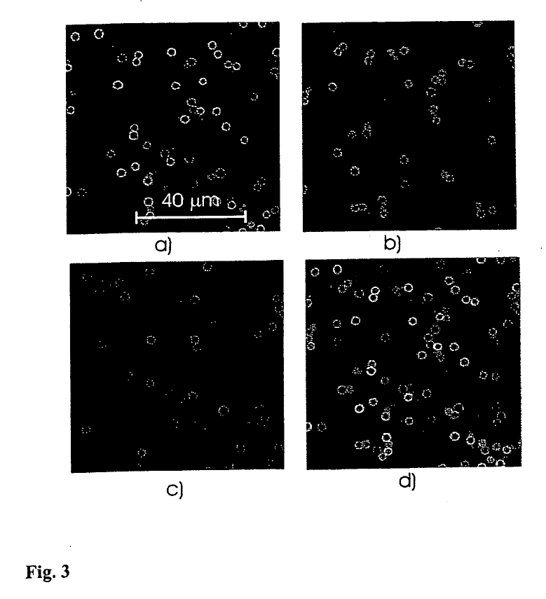

The capsules were investigated by means of confocal laser scanning microscopy while simultaneously using 3 different channels (FIGS. 3a-c). The excitation wavelength of the lasers was 488 nm in the case of fluorescein, 543 nm in the case of rhodamine and 633 nm in the case of Cy5. The detectors were set to maximum emission of the dyes and to a minimal overlap of their fluorescence emissions. The laser intensities and the detector sensitivities were adjusted to approximately equal signal intensities for each channel. Superimposition of the 3 channels showed 7 differently colored capsules (FIG. 3d).

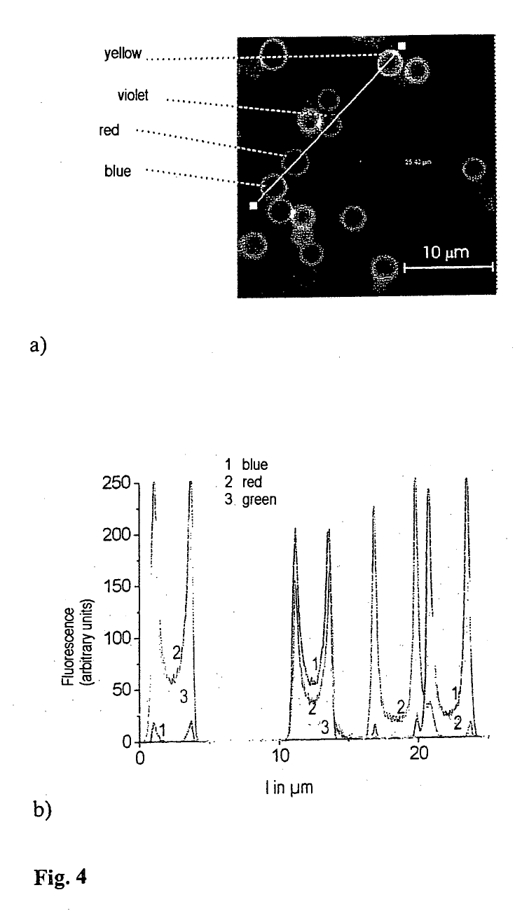

Analysis of the fluorescence intensities along a profile through the capsules provides a quantitative and reliable method for distinguishing between the different capsules. The profiles show the distribution of the fluorescence intensities of different channels for the same capsule. FIG. 4a shows, for example, the profile of capsules 2, 7, 1 and 5.

The fluorescence intensities per dye layer are different for differently colored capsules, a fact which can be attributed to resonance energy effects and different contents of adsorbed material. The resonance energy transfer can be markedly reduced by using several layers between the dye layers. Above a distance of 6 nm (approx. 4 layers), there are virtually no interactions any longer between the dye molecules.

Controlled Förster Resonance Energy Transfer

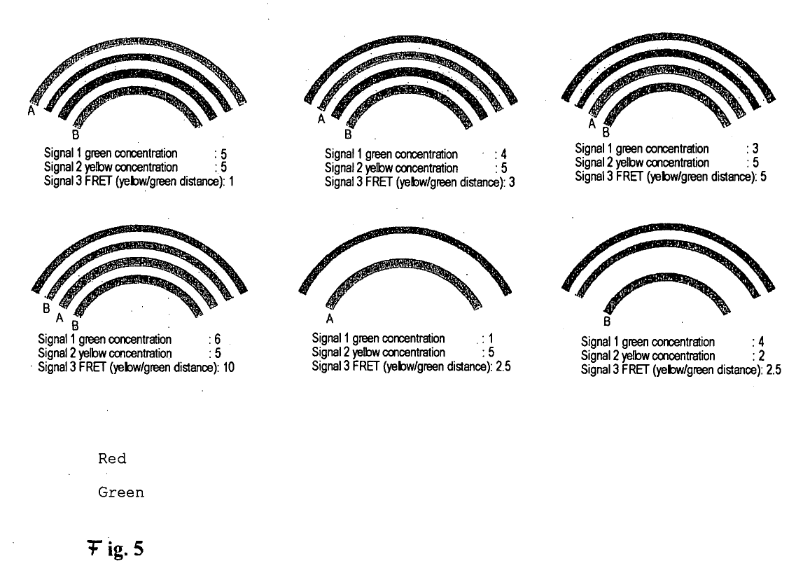

In order to use fixed distances between the dye molecules for the purpose of protecting trademarks against forgery, capsules were prepared which possessed different distances between the dyes but the same content of dye. FIG. 5 shows the layer combinations which were prepared.

The information encoded in the capsules by two dyes can be determined by using two different excitation wavelengths and measuring fluorescence at two different wavelengths. In the case of the rhodamine/fluorescein system this means:
1. Excitation light at 540 nm, measurement of the emission at 576 nm: this gives the absolute concentration of rhodamine
2. Excitation light at 495 nm, measurement of the emission at 520 nm: this gives the concentration of fluorescein minus the concentration of the molecules which are undergoing an energy transfer to rhodamine
3. Excitation light at 495 nm, measurement of the emission at 576 nm: this gives the intensity of the FRET or the mean distance between the dye molecules (forgery detection)

Each of the capsule types prepared gives a specific ratio between signal 1:signal 2:signal 3. For measuring small differences in the signal intensity, these two dyes are already sufficient for realizing a large number of coding possibilities. However, the number of the dyes in capsules can be up to 7.

Using Förster Resonance Energy Transfer for Sensory Applications

Capsules 2 and 3 from table 1 were used for the sensor applications. We found that, depending on chain length, PAH/PSS layers swell strongly or shrink when solutions of quaternary alkyl ammonium salts are added. (PAH/PSS)₅ capsules are found to swell strongly, from 3 μm up to 5.7-6.0 μm, when a 0.05 M solution of dodecyltrimethylanmonium bromide (DODAB) is added. When the capsule diameter is doubled, the distance between the dye layers will also double, when the layers swell isotropically, whereas the volume of a layer increases by a factor of 8.

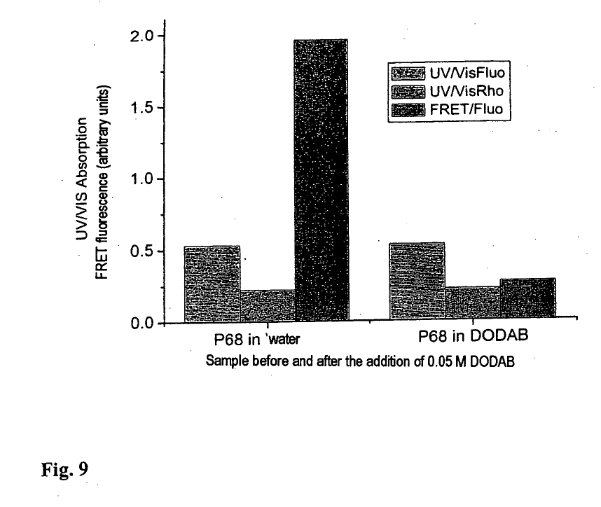

Capsule 2 was used in experiment 1. The concentration of rhodamine and fluorescein in the capsule wall was determined UV/VIS-spectroscopically before and after the swelling process. The mean distance between the two dye layers was about 4.5, nm before the treatment and almost 9 nm after the treatment. The change in the FRET signal (λ_{exc}=495 nm, λₑₘ=578 nm) was monitored during the swelling process using a fluorescence spectrometer (FIG. 9). As a result of the swelling of the layers, the intensity of the FRET signal decreased by 86% during the reaction with 0.05 M DODAB.

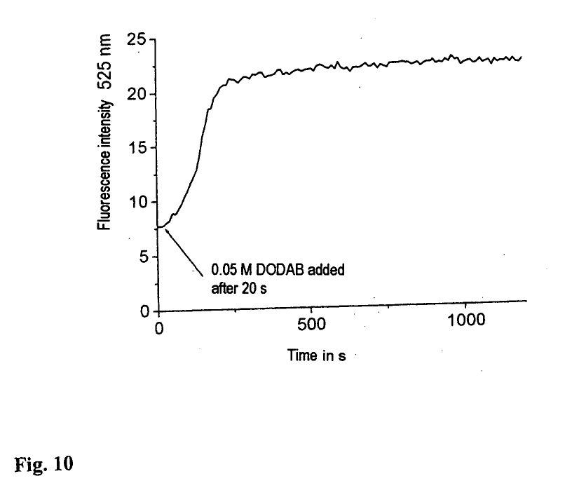

Capsule type 3 was used in experiment 2. An efficient quenching process occurs as a result of the high concentration of fluorescein in the one PAH layer. After 0.05 M DODAB solution has been added, the volume of the PAH layer increases by about a factor of 8. As a result of the decrease in the self-quenching of the dye, the fluorescence of the capsules thereby increases by 290% (FIG. 10).

Filling the Capsules with Reactive Macromolecules:

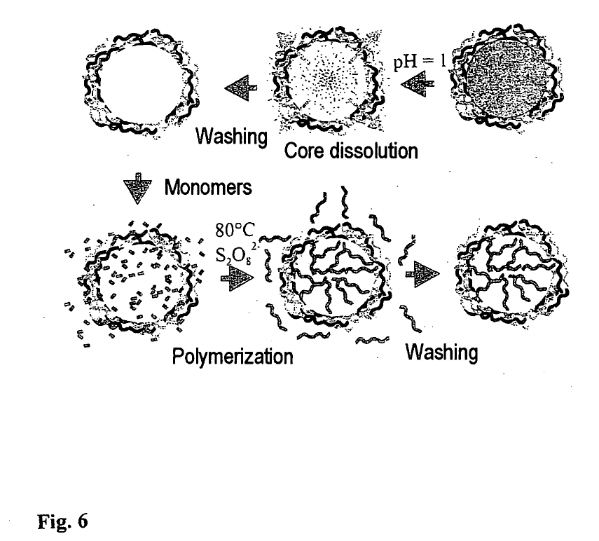

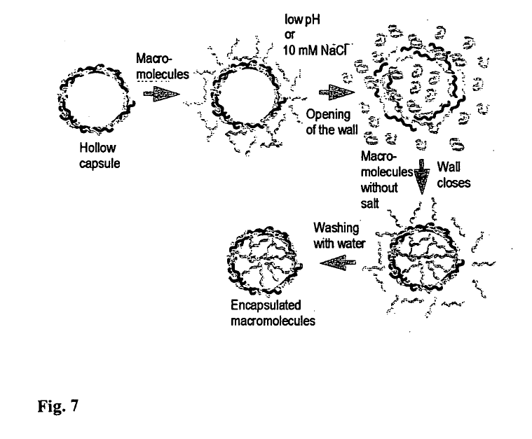

There are three different ways for immobilizing macromolecules in the interior of the capsules:
1. “Ship in bottle” synthesis of polymers within the capsules (FIG. 6).¹²
2. Using salts or pH changes to switch the permeability of specific capsules for corresponding macromolecules (FIG. 7)¹¹
3. Forming a precipitate of an unstable complex, composed of the macromolecules and an auxiliary substance, on the colloidal template. Subsequently encapsulating the material by means of the customary LbL method and dissolving the core and the macromolecular complex.⁸

Other advantageous embodiments of the capsules according to the invention, and of their use, are cited below, with it being possible to combine all the embodiments with each other at will:
Capsules which are prepared from polyelectrolyte multilayers in accordance with the layer-by-layer method and which are smaller than 100 μm, for coding and sensory/diagnostic/analytical applications, and which contain
   a) a defined assignment of dye-labeled polyelectrolytes to the layer number,
   b) a defined assignment of dye-free polyelectrolytes to the layer number,
   c) a defined assignment of sensory polyelectrolytes or sensorially reactive coating components to the layer number
   d) a defined assignment of interactions of the labels of different layers
Capsules, with core or without core, as envelopes which contain the solvent or a solution of a different composition.
Capsules which contain one or more fluorescent dyes in at least two layers which make it possible to adjust, in a defined manner, both the fluorescent colors and their intensities and the interactions or self-interactions.
Capsules which contain at least two fluorescent dyes in different layers, which dyes are linked to each other by way of Förster resonance energy transfer (FRET).
Capsules which contain at least one sensory intermediate layer which is located between FRET-capable donor and acceptor fluorescent dye-labeled layers and which, in adaptation to changed properties of the medium, e.g. pH, salt concentration, temperature, adsorbed components, enzymes, and miscible solvent constituents and components which affect the solvent or react with the intermediate layer, influences the FRET signal in a measurable manner and can be used as a sensor for this change.
Capsules which contain at least two fluorescent dyes whose distance from each other suppresses the Förster resonance energy transfer.
Capsules with at least one layer which contains a fluorescent dye at a density which can lead to self-interaction (self-quenching) within the layer and which can be influenced in a measurable manner by changes of components or conditions within the medium or the environment and can serve as a sensor for these components or conditions.
Capsules, with the capsules being smaller than 10 μm, preferably smaller than 1 μm.
Capsules which contain a modified core which can possess sensory functions or coding properties.
Use of the capsules as a library of reporter particles or coded color particles for identifying substances and/or labeling processes.
Use of the capsules in medical diagnosis, combinatorial chemistry, genomics and proteomics, biology and biotechnology and industry.
Use of the capsules for coding industrial products.
Use of the capsules for labeling particles, cells, tissues, organs and organisms of biological origin.
Composition for identifying substances, with the composition comprising at least two types of capsule having diameters of less than 100 μm, with the capsules possessing a core and an envelope and the envelope having at least three layers, with at least one of these layers being labeled with a dye.
Composition which comprises at least 3 types of capsule.
Composition, with the capsules possessing an average diameter of less than 10 μm, preferably less than 1 μm.
Composition with the envelopes being composed of polyelectrolyte layers.
Composition, with at least one capsule type being defined by capsules whose envelopes are composed of at least two layers which are labeled with different dyes, with the layers which are labeled with different dyes being separated from each other by at least one layer which is not labeled with dyes.

FIGS. 1 to 10 show various embodiments of the invention.

FIG. 1 shows the structure of the fluorescent dyes used.

FIG. 2a) shows the absorption spectrum (normalized intensity), and FIG. 2b) shows the fluorescence spectrum (normalized intensity), of PAH-Fl, PAH-Rho and PAH-Cy5.

FIG. 3 depicts confocal images of a mixture of color-coded capsules. 3a) shows the fluorescein channel, i.e. the fluorescence of fluorescein, while 3b) shows the rhodamine channel 3c) shows the Cy5 channel and 3d) shows the superimposition of the three color channels.

FIG. 4 shows a mixture of colored capsules 2, 7, 1 and 5. A confocal fluorescence microscope was used for the photographs. The superimposition image of the three color channels of the fluorescence microscope can be seen in FIG. 4a), while the profile of the fluorescence intensity along the white line in FIG. 4a) can be seen in FIG. 4b).

FIG. 5 makes clear the principle of construction of the layer combinations prepared, with figs. a-c) showing different FRET signal intensities in association with the same dye concentration and FIGS. 5d-f) showing different FRET signal intensities in association with different dye concentrations, with a) being located at the top left and f) being located at the bottom right.

FIG. 6 depicts the principle of the steps of the so-called “ship-in-bottle” synthesis of polymers within the capsules. After the core which has been used as a template for the coating with the polyelectrolytes has been dissolved away, monomers pass through the envelope and arrive in the interior of the capsule. Under suitably selected conditions, the monomers polymerize and can therefore no longer pass through the envelope. In a concluding washing step, the polymers located outside the capsules are removed from the solution. The encapsulated monomers remain behind.

FIG. 7 shows the principle of loading MF capsules (8 layers) by means of using salt or the pH to switch the permeability of special capsules for corresponding macromolecules. The pores of the envelopes can be enlarged, and the permeability thereby increased, by altering the salt content and/or the pH. This enables even relatively large macromolecules to penetrate into the capsules. In conclusion, the pH and/or the salt content is returned once again to the initial values; the pores close once again or become smaller. The macromolecules which have penetrated into the capsules can no longer pass through the envelope.

FIG. 8 shows a diagram of the construction and mode of action of the two different sensor capsules which are described above. The upper row in FIG. 8 depicts capsule 2 while the lower row depicts capsule 3. Adding DODAB increases the thickness of the unlabeled intermediate layer (sensitive layer) such that the distance between the two labeled layers increases. This decreases the coupling between the dyes, resulting in the FRET being weaker. As a consequence, the fluorescence of the donor dye which is registered at 578 nm is lower.

FIG. 9 depicts the signal intensities of capsule No. 2

a) in water, and

b) after a 0.05 M DODAB solution has had its effect. (green absorption of the fluorescein at 495 nm, red absorption of the rhodamine at 553 nm, and blue FRET signal λ_{exc}=495 nm, λₑₘ=578 nm)

In comparison, the fluorescence intensity of capsules No. 3 following the addition of 0.05 M DODAB is depicted in FIG. 10.

### REFERENCES

1. Battersby, Bronwyn Jean et al. Patent WO 00/32542, June 2000
2. Payan, Donald US Patent 20010006787, A1, July 2001,
3. Still, et al. U.S. Pat. No. 5,565,324, October 1996; Still et al., U.S. Pat. No. 6,001,579, March 1999
4. Norrman, Nils, Patent EP 1190256, March 2002
5. Trau, Mathias et al. WO 99/24458, May 1999
6. Donath, E. et al., WO 99/47252, March 1999
7. Spiro, A.; Lowe, M.; Brown, D. Appl. Env. Microbiology 66, 2000, 4258
8. Gaponik, N., Radtchenko, I. L., Sukhorukov, G. B., Weller, H., Rogach, A. L. Adv. Mater. 14, 2002, 879
9. E. Donath, G. B. Sukhorukov, F. Caruso, S. A. Davis, H. Möhwald, Angew. Chem. Int. Ed. 1998 37, 2002.
10. R. Steitz, V. Leiner, R. Siebrecht, R. V. Klitzing, Colloids a. Surf. A, 2000, 163, 63.
11. G. Ibarz, L. Dähne, E. Donath, H. Möhwald “Smart Micro- and Nanocontainers for Storage, Transport and Release” Adv. Mater. 13 (2001) 1324-1327.
12. L. Dähne, E. Donath, S. Leporatti, H. Möhwald, “Synthesis of micro reaction cages with defined chemical properties” J. Amer. Chem. Soc. 123 (2001) 5431-5436.
13. H. Härmä, “Particle technologies in diagnostics” TEKES Technology Review 126/2002.

1-25. (canceled)

26. A capsule, comprising:
an envelope having a diameter of less than 100 μm, and
the envelope comprising at least three polyelectrolyte layers, with at least one of these three polyelectrolyte layers being labeled with at least one dye.

27. The capsule as claimed in claim 26, wherein two of the three polyelectrolyte layers are in each case labeled with different dyes, with the two polyelectrolyte layers which are labeled with the different dyes being separated from each other by at least the third polyelectrolyte layer which is not labeled with dyes.

28. The capsule as claimed in claim 27, wherein the third polyelectrolyte layer, which is not labeled with dyes, has a thickness of between 0.1 nm and 10 nm.

29. The capsule as claimed in claim 28, wherein the third polyelectrolyte layer, which is not labeled with dyes, is a sensitive layer which either swells or shrinks, with its thickness thereby being altered, when its environmental conditions change.

30. The capsule as claimed in claim 29, wherein the environmental conditions are pH, salt concentration, and temperature.

31. The capsule as claimed in claim 27, wherein the different dyes are a dye of higher absorption energy (donor) and a dye of lower absorption energy (acceptor).

32. The capsule as claimed in claim 31, wherein the different dyes are coordinated with each other such that it is possible for a Förster (fluorescence) resonance energy transfer (FRET) to take place between the different dyes.

33. The capsule as claimed in claim 27, wherein additional polyelectrolyte layers, which are not labeled with dyes, are located between the polyelectrolyte layers which are labeled with the different dyes.

34. The capsule as claimed in claim 29, wherein the sensitive layer is an organic polyelectrolyte layer.

35. The capsule as claimed in claim 26, wherein the dye is covalently linked, at high concentration, to a sensitive material.

36. The capsule as claimed in claim 35, wherein the sensitive material is a material which either swells or shrinks, with its volume thereby being altered, when its environmental conditions change.

37. The capsule as claimed in claim 36, wherein the environmental conditions are pH, salt concentration, and temperature.

38. The capsule as claimed in claim 35, wherein the concentration of the dye is so high that the dye forms dimers, aggregates or excimers with itself, which latter lead to self-quenching of the fluorescence or to the formation of a new emission band.

39. The capsule as claimed in claim 35, wherein the concentration of the dye satisfies the relationship mass of sensitive material:mass of dye <500:1.

40. The capsule as claimed in claim 35, wherein the dye-labeled layer has a thickness of from 1 nm to 1 μm.

41. The capsule as claimed in claim 35, wherein the polyelectrolyte layer which is labeled with dyes is an organic polyelectrolyte layer which is labeled with dyes.

42. The capsule as claimed in claim 26, wherein the dyes are fluorescent dyes or emitting nanoparticles.

43. The capsule as claimed in claim 26, wherein the capsule is hollow and macromolecules are located within the internal space which is delimited by the envelope.

44. The capsule as claimed in claim 26, wherein the envelope is permeable to molecules of up to a given size.

45. The capsule as claimed in claim 26, wherein the capsule possesses a solid core which is surrounded by the envelope.

46. The capsule as claimed in claim 26, wherein the capsule has an average diameter of less than 10 μm.

47. The capsule as claimed in claim 26, wherein the capsule is prepared by the layer-by-layer method.

48. The capsule as claimed in claim 26, wherein the capsule is used for labeling or coding industrial products, particles, cells, tissues, organs or organisms of biological origin.

49. A composition for identifying or labeling substances, comprising at least two types of different capsules as claimed in claim 1.

50. The composition as claimed in claim 49, comprising at least three types of different capsules as claimed in claim 1.
